(19) United States
(12) Patent Application Publication
(10) Pub. No.: US 2026/0354674 A1
(43) Pub. Date: Nov. 12, 2026

(54) A URETEROSCOPE

(71) Applicant: BIORAD MEDISYS PRIVATE LIMITED, Pune (IN)

(72) Inventors: Debasish PRADHAN, Pune (IN); Anshuman MATHUR, Pune (IN); Aman SAXENA, Pune (IN); Saurabh SINGH, Pune (IN); Giriraj OZA, Pune (IN)

(21) Appl. No.: 19/486,958

(22) PCT Filed: May 24, 2024
(86) PCT No.: PCT/IN2024/050611

§ 371 Date: Nov. 24, 2025

(30) Foreign Application Priority Data
May 24, 2023 (IN) 202321035889

(51) Int. Cl. A61B 1/307; A61B 1/00; A61B 1/005; A61B 1/05

(52) U.S. Cl. CPC A61B 1/307; A61B 1/00009; A61B 1/00128; A61B 1/0016; A61B 1/0052; A61B 1/0057; A61B 1/05

(57) ABSTRACT

An ureteroscope (100) comprises a handle assembly (102a) and a tube assembly (102b). The handle assembly (102a) comprises a casing (302) and a motor (310). The casing (302) comprises a knob (304) and a rotary encoder (308) connected to the knob (304). One end of the motor (310) is connected to the rotary encoder (308) and other end of the motor is connected with a driving pin (318). The tube assembly (102b) is coupled to the handle assembly (102a). The tube assembly (102b) comprises a differential gear mechanism (406) and a plurality of wires (412). The differential gear mechanism (406) comprises a pair of meter gears (408) and a meter pinion (410). The meter pinion is engaged with the driving pin (318). During an operation, the bending section (112) is articulated based on a rotation of the knob (304).

### TECHNICAL FIELD

The present invention relates generally to a field of medical instruments. More specifically, the present invention relates to an endoscopic device particularly a ureteroscope having disposable and reusable components and method of assembling them.

### BACKGROUND

Endoscopic devices for surgical or diagnostic procedure are already known. Endoscopes have gained widespread acceptance in the medical community because they provide a means for performing surgery with minimal trauma to the patient while allowing the physician to view internal anatomy of the patient. Over the years, many endoscopes have been developed and classified according to specific applications, such as ureteroscopes, cystoscopes, colonoscopes, laparoscopes, upper gastrointestinal endoscopes, and the like. The endoscope may be inserted into a natural opening in the body, or through an incision in the skin. A conventional endoscope is generally an instrument with a handle and an insertion part wherein, various working components for observation like an optical or electronic imaging system and the like are provided at a distal end of the insertion part, a proximal end at handle with controls for manipulating the tool and devices for viewing the image and a solid or tubular elongate shaft connecting the ends is provided.

To use an endoscope, a surgeon inserts the distal end into a patient through a natural orifice or an artificial incision to diagnose or treat a disease state of the patient using the working components at a distal end of the insertion part. The proximal end remains outside the patient and typically connects to an eyepiece, video monitor, or other equipment. The insertion part uses a complex lens system for transmitting the image from the distal tip of the endoscope to a viewer. The maneuverability of an endoscope and its suitability for accessing small and delicate regions of the body are in part determined by the size of the endoscope tip, which in turn is determined by the sizes of various components the tip contains. It is desirable that the distal end part of an endoscope should be as thin as possible because the thinner the distal end part, the more effectively it can be inserted into a hollow organ of the patient's body, so that the pain inflicted on the patient is minimized. Endoscopes may also incorporate additional features for observation or operation within the body, such as a working channel for passing diagnostic, monitoring, treatment, or surgical tools through the endoscope, where the working channel has an opening located at the distal end portion of the endoscope to provide access to a site within the patient.

In the case of both rigid and flexible conventional endoscopes, the lens or fiber optic system is relatively expensive. In more currently developed endoscopes, discrete illuminators such as Light-Emitting Diodes (LEDs) have been incorporated for providing illumination. Currently available endoscopes have drawbacks, especially for navigating through a passageway of a patient. A common problem encountered in such procedures is obscuring of the examination field. This is typically caused by bleeding or release of other fluids from the excision site, where the blood or other fluid released prevents the user from clearly observing the examination field. In addition, laser lithotripsy operation needs to be performed for crushing kidney stones in a water-containing environment (such as saline, contrast agents, etc.) the use of water in the operation can cause the change of the internal pressure of the organ, and can cause great harm to the human body when the internal pressure of the organ is too high or too low. High intraluminal pressure, in some instances, may make it easier for bacteria and endotoxins to be absorbed into the blood, possibly resulting in postoperative fever. High pressure may also cause lymph node and venous reflux which may lease to fluid leakage, post-operative pain, urosepsis and renal injury. Further, rapidly rising intrarenal pressures may increase the risk of extravasation with the potential to cause pyelosinus, pyelovenous, and/or pyelolymphatic backflow of an irrigant provided to the kidney which severely endangers the life of the patient.

As disposable endoscopy is an emerging category of endoscopic instruments, in some cases the manufacture of single use endoscopes can be expensive enough to be used on a single patient only, because after a single use the whole device is thrown eventually increasing material wastage. Though, disposable or single-use endoscopy lessens the risk of cross-contamination and hospital acquired diseases, but sometimes these single use endoscopes are not disposed and used multiple times on different patients. In order to use the same endoscope multiple times, after an endoscope is used on a particular patient, the endoscope must be sterilized before it can be used again. The goal of sterilization is to remove all foreign matter and all pathogens. However, it is difficult to clean and disinfect thoroughly every time which leads to potential problems of cross-contamination between patients and between patients and staff.

While the state-of-the-art relating to endoscopes is relatively advanced today, there are still recurring problems and to overcome the above problems, there is a need for split type disposable endoscopes wherein the part of the endoscope which is in direct contact with the patient fluids is disposed of and the part which is not in contact can be reused again and again. This invention is therefore directed towards providing an improved split type endoscope, which is relatively simple in design and structure, and is highly effective for its intended purpose.

### OBJECTIVES

An object of the present invention is to provide an improved ureteroscope with a combination of disposable and reusable components which can be easily used by the physician.

Another object of the present invention is to provide an improved ureteroscope having a disposable part adapted to detachably engage to a reusable part, such that an interface or operative connection is established between the internal components of both reusable and disposable part when engaged together.

Yet another object of the invention is to provide a ureteroscope which is simple in structure, easy to attach and detach, easy and convenient to operate, saves operation time and high in working efficiency.

Still another object of the invention is to provide an improved ureteroscope tip structure for traumatically navigating a passageway that resolves or improves on one or more of the above-described drawbacks.

Another object of the invention is to provide an improved ureteroscope having a tip structure of a ureteroscope with a camera and LED module for illumination thereby eliminating fiber optics cable.

### SUMMARY

Embodiments of the present disclosure present technological improvements as solutions to one or more of the above-mentioned technical problems.

Before the present subject matter relating to a ureteroscope, it is to be understood that this application is not limited to the particular system described, as there can be multiple possible embodiments which are not expressly illustrated in the present disclosure. It is also to be understood that the terminology used in the description is for the purpose of describing the implementations or versions or embodiments only and is not intended to limit the scope of the present subject matter.

This summary is provided to introduce aspects related to the ureteroscope. This summary is not intended to identify essential features of the claimed subject matter nor is it intended for use in determining or limiting the scope of the present subject matter.

In an embodiment of a present invention, a ureteroscope is disclosed. The ureteroscope comprises a handle assembly and a tube assembly. The handle assembly comprises a casing and a motor. The casing comprises a knob and a rotary encoder connected to the knob. One end of the motor is connected to the rotary encoder and other end of the motor is connected with a driving pin via a shaft. The tube assembly is coupled to the handle assembly. The tube assembly comprises a differential gear mechanism and a plurality of wires. The differential gear mechanism comprises a pair of meter gears and a meter pinion. The meter pinion is engaged with the driving pin. A first end of each of the wires is connected with the pair of meter gears and a second end of each of the wire is connected with a bending section. During an operation, the bending section is articulated based on a rotation of the knob.

This summary is provided to introduce aspects related to a ureteroscope. This summary is not intended to identify essential features of the claimed subject matter nor is it intended for use in determining or limiting the scope of the present subject matter.

Additional aspects, advantages, features and objects of the present disclosure would be made apparent from the drawings and the detailed description of the illustrative embodiment.

It will be appreciated that features of the present disclosure are susceptible to being combined in various combinations without departing from the scope of the present disclosure as defined by the below mentioned detailed description and drawings.

### BRIEF DESCRIPTION OF THE ACCOMPANYING DRAWINGS

The foregoing detailed description of embodiments is better understood when read in conjunction with the appended drawings. For the purpose of illustrating the disclosure, there is shown in the present document example constructions of the disclosure; however, the disclosure is not limited to the specific system or method disclosed in the document and the drawings.

The present disclosure is described in detail with reference to the accompanying figures. In the figures, the left-most digit(s) of a reference number identifies the figure in which the reference number first appears. The same numbers are used throughout the drawings to refer various features of the present subject matter.

FIG. 1(a) and 1(b) illustrate the assembled and disassembled view of a ureteroscope to observe internal organ of a patient with a Vision Processing Unit (VPU) in accordance with an exemplary embodiment of the present disclosure.

FIG. 2 illustrates the handle assembly with bevel gear mechanism for articulation of bending section in accordance with an exemplary embodiment of the present disclosure.

FIG. 3 illustrates the handle assembly with rotary encoder and motor for articulation of bending section in accordance with another embodiment of the present disclosure.

FIG. 4(a) and 4(b) illustrate an exploded view of disposable handle assembly in accordance with an exemplary embodiment of the present disclosure.

FIG. 5 illustrates perspective view of bending section in accordance with an exemplary embodiment of the present disclosure.

FIG. 6 illustrates exploded view of distal tip present at the distal end of the tube assembly of the ureteroscope in accordance with an exemplary embodiment of the present disclosure.

FIG. 7 illustrates perspective view of Vision Processing Unit (VPU) in accordance with an exemplary embodiment of the present disclosure.

In the above accompanying drawings, a non-underlined number relates to an item identified by a line linking the non-underlined number to the item. When a number is non-underlined and accompanied by an associated arrow, the non-underlined number is used to identify a general item at which the arrow is pointing.

Further, the figures depict various embodiments of the present subject matter for purposes of illustration only. One skilled in the art will readily recognize from the following discussion that alternative embodiments of the structures and methods illustrated herein may be employed without departing from the principles of the present subject matter described herein.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although a ureteroscope, similar or equivalent to those described herein can be used in the practice or testing of embodiments of the present disclosure, the exemplary, a ureteroscope is now described.

For purposes of the present specification and claims, various relational terms like “top” “bottom,” “proximal,” “distal,” “upper,” “lower,” “front,” and “rear” are used to describe the present invention when the invention is positioned in or viewed from a given orientation. It is to be understood that, by altering the orientation of the invention, certain relational terms may need to be adjusted accordingly.

The person skilled in the art will recognize many variations, alternatives, and modifications of the embodiment of the present disclosure. It should be understood that this invention is not limited to the particular methodology, protocols, and the like, described herein and as such may vary. The terminology used herein is for the purpose of describing particular embodiment only and is not intended to limit the scope of the present invention, which is defined solely by the claims.

Various modifications to the embodiment will be readily apparent to those skilled in the art and the generic principles herein may be applied to other embodiments. For example, although the present disclosure will be described in the context of a ureteroscope, one of ordinary skill in the art will readily recognize that a system can be utilized to reduce cost and material wastage by including a partially disposable and remaining usable part in the design of ureteroscope. Thus, the present disclosure is not intended to be limited to the embodiments illustrated but is to be accorded the widest scope consistent with the principles and features described herein.

To use a ureteroscope, a surgeon inserts the distal end of the ureteroscope into a patient's body through a natural orifice or an artificial incision to diagnose or treat a disease state of the patient. In order to avoid cross contamination, the endoscope must be sterilized before it can be used again to remove all foreign matter and all pathogens. However, it is difficult to clean and disinfect thoroughly every time which leads to potential problems of cross-contamination between patients and between patients and staff. Therefore, discarding the device after single use is recommended. However, manufacturing of single use ureteroscope can be expensive enough to be used on a single patient only. Also, disposing the whole ureteroscope eventually increases the material wastage. The present invention discloses a ureteroscope which is a split type disposable ureteroscope wherein the part of the ureteroscope which is in direct contact with the patient's fluid is disposed of and the part which is not in contact with the patient's fluid can be reused again and again.

The embodiment of the present disclosure provides an ureteroscope configured with a combination of disposable and reusable components solving the problem of high renal pressure in the kidney by placing sensor to control the parameters and acts as assistive feedback to the user. The present disclosure discloses a solution for solving the problem of stone management.

In an embodiment, a ureteroscope is disclosed. The ureteroscope comprises a handle assembly and a tube assembly. The handle assembly is a reusable assembly and the tube assembly is a disposable assembly which is used to insert in a person's body for diagnosing or treating a disease state of the patient. The handle assembly comprises a casing and a motor. The casing comprises a knob and a rotary encoder. The knob is an articulation knob adapted to control the articulation of a bending section at desired angle with the help of a motor mechanism. The rotary encoder is connected with the knob. When knob is rotated from its position between its specified angle range, feedback of this movement is given to the motor encoder/stepper motor. According to the feedback, the motor encoder/stepper motor rotates either in clockwise or anti-clockwise direction.

The casing comprises a socket at a distal end of the handle assembly. The socket is cylindrical in shape. The socket comprises a plurality of pogo pins and a driving pin. Pogo pins are spring loaded electrical connection pins. The driving pin is a projection in the shape of a tapered hexagonal. One end of the motor is connected to the rotary encoder and other end of the motor is connected with a driving pin via shaft. The socket comprises a geometry adapted to lock with the tube assembly.

The tube assembly is coupled to the handle assembly. The tube assembly comprises tube casing with a working channel, an insertion tube, a bending section and a distal tip. The tube casing comprises a differential gear mechanism and a plurality of wires. The differential gear mechanism used in this disclosure is more precise and reliable. The differential gear mechanism comprises a pair of meter gears and a meter pinion. The meter pinion is engaged with the driving pin. A first end of each of the wires from a plurality of wires is connected with the pair of meter gears and a second end of each of the wires is connected to a bending section. Second end of each of the wires is welded with the bending section. In an embodiment, the plurality of wires is made up of steel. The plurality of wires passes through the tube assembly and wound around the pair of gears on the other end. One end of the meter pinion comprises a tapered hexagonal cavity and other end of the meter pinion is engaged with the pair of meter gears.

The driving pin's hexagonal tapered projection from the handle assembly is positively coupled with the tapered hexagonal cavity in the meter pinion via a bearing adapted to be at the centre of a pogo pin connector. The pogo pin connector comprises same number of contact pads as that of plurality of pogo pins present on the handle assembly to transfer the electrical signals. The tube casing also comprises a spring based lock that helps in quick engagement and disengagement of the handle assembly. The main function of electromechanical coupling is to transfer mechanical motion and electronic signals for image, lighting, pressure etc.

The tube assembly comprises a spring based locking mechanism that helps to easily attach and detach the handle assembly. The locking mechanism comprises a lock and a push button. The ureteroscope has a coupling mechanism such that the handle assembly and the bending section of the tube assembly can be mutually attached and detached from each other wherein the coupling mechanism establishes an electrical interface/connection between the internal components of the handle assembly and internal components of the bending section. The lock is easy to use, and is activated by pressing the push button against the spring load, which in turn creates the suitable space for a handle male geometry in the tube assembly. When the handle comes at the desired location, push button is released, the spring on the button provides sufficient force that helps to engage the geometry present on the push button with the geometry on the handle assembly. This arrangement locks the handle assembly and the tube assembly together. The lock ensures firm attachment and proper functioning during usage.

During operation, the knob is rotated from its position between its specified angle range. Feedback of this movement is given to the motor encoder/stepper motor. According to the feedback, the motor encoder/stepper motor rotates either in clockwise or anti-clockwise direction and torque is transferred to the bending section thereby articulating the bending section using differential gear mechanism through the plurality of wires. The differential gear mechanism helps to control pulling the plurality of wires more efficiently. The differential gear mechanism helps to self-align the tube assembly and bring it back to the home position which makes easy attach and detach. The technology used in the present invention provides self-positional locking so that the bending section can stay at the same position when articulated. This helps to reduce the stress level on the user hands.

The distal tip comprises a smallest trapezoidal shaped camera and LED module arrangement that helps in building a 7.5 Fr ureteroscope, a working channel passage, a camera tip, and a pressure sensor on the camera tip. The pressure sensor and the camera are connected to the timer PCB 422 present in the tube assembly by electrical wires. When the tube assembly and the handle assembly are attached to each other, pogo pins and contact pads come in contact and the image signal(s) from the camera and the signals from the pressure sensor are transferred.

The ureteroscope comprises a cable. One end of the cable is connected to the handle assembly and other end of the cable is connected to a Vision Processing Unit (VPU) via a connector. The VPU comprises a receptacle PCB to receive one or more images from the camera in RGB format, and then performs combination of image processing steps to enhance the image quality. Dedicated image processing devices are typically used to deliver optimal image quality at high resolution with minimal lag. The VPU also comprises a processor board to manage data flow and to control the algorithms and functions of the PCB. VPU further comprises a screen mainly used to display the image or video captured by the camera. It also has some options to control white balance, sharpness etc. It is also common to have multiple display monitors for a single system, where one or multiple display monitors are connected for viewing or control by Surgeons.

It should be noted that the above advantages and other advantages will be better evident in the subsequent description. Further, in the subsequent section the present subject is better explained with reference to the figures.

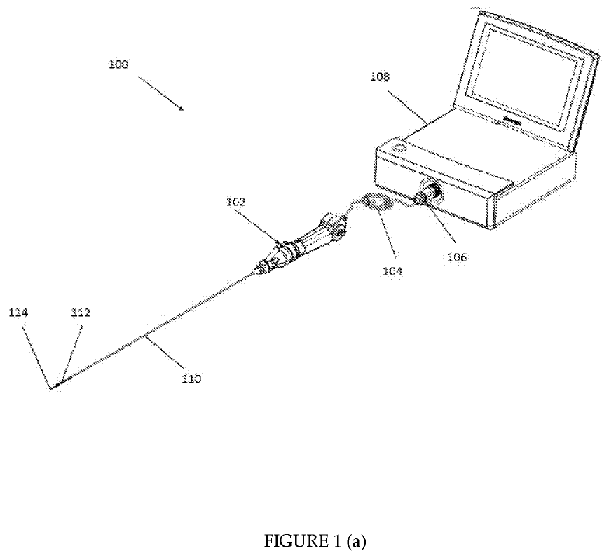

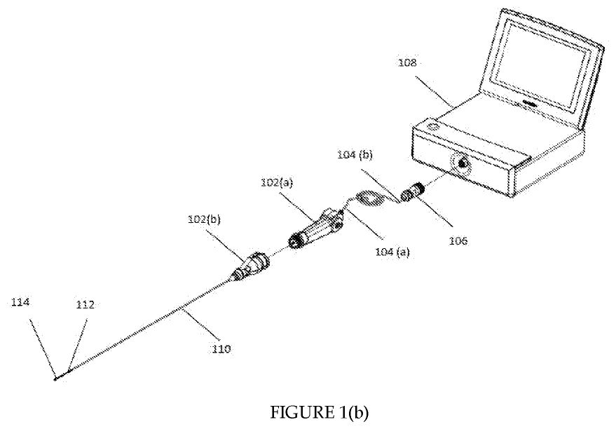

Referring now to the drawings, FIGS. 1-7 illustrate a ureteroscope to observe an internal organ according to present invention. It should be noted that FIG. 1-7 are merely examples. A person skilled in the art will recognize many variations, alternatives, and modifications of the embodiments of the present disclosure.

Referring now to the drawings, FIG. 1(a) and 1(b) illustrate the assembled and disassembled view of a ureteroscope 100 respectively to observe internal organ of a patient for minimally invasive surgery (MIS) particularly urology, is disclosed according to an exemplary embodiment of the present invention. A ureteroscope 102 comprises a handle assembly 102a and a tube assembly 102b. The handle assembly 102a is a reusable assembly and the tube assembly is a disposable assembly, as the tube assembly is used for insertion. The ureteroscope 100 further comprises a cable, wherein the cable is a scope handle cable 104. One end of the cable 104 is connected to the handle assembly 102(a) and other end of the cable 104 is connected to the Vision Processing Unit (VPU) 108 via a connector 106. The tube assembly 102(b) comprises insertion tube 110 having a bending section 112 with a distal tip 114 at the distal end of the ureteroscope 102.

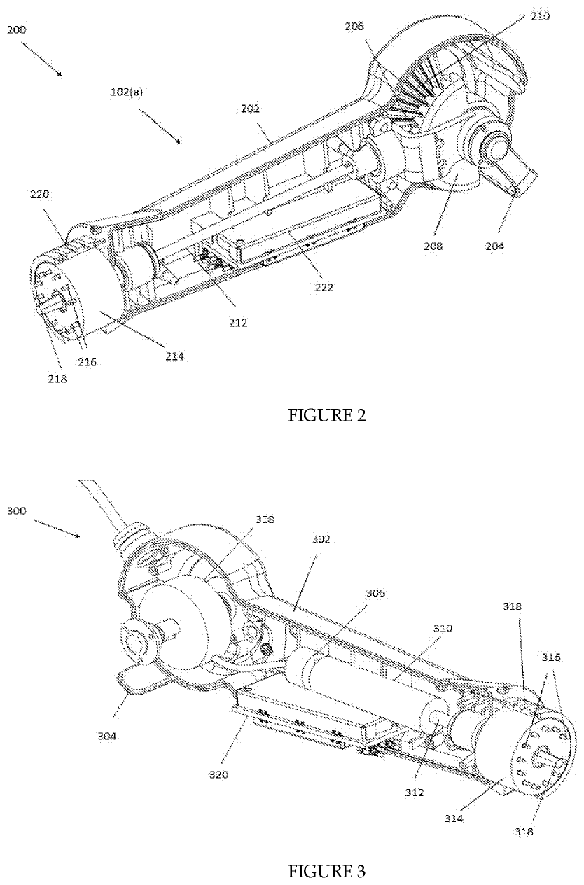

Referring to FIG. 2, an embodiment for a handle assembly 102a is disclosed. The handle assembly 102a comprises a casing 202 having an articulation knob 204 adapted to control the articulation of bending section 112 at desired angle with the help of a bevel gear mechanism 206. The casing 202 further comprises a socket 214 at a distal end of the handle assembly 102a. Socket is a cylindrical part. The socket 214 comprises a plurality of pogo pins 216 and a driving pin 218. Each of the pogo pin 216 from the plurality of pogo pins 216 is a spring loaded electrical connection pin. The driving pin 218 is a projection in the shape of a tapered hexagon. Further, the socket 214 comprises a geometry 220 adapted to lock with the tube assembly 102b. The tube assembly 102b comprises spring based locking mechanism that helps to easily attach and detach the handle assembly 102a and tube assembly 102b. The casing 202 further comprises a PCB with RF shielding 222.

The knob 204 at the handle assembly 102a is connected with a bevel pinion 208 in a handle housing 224. Bevel gear 210 is engaged with bevel pinion 208. The bevel gear 210 is further connected with one end of a straight driving shaft 212 and other end of the straight driving shaft has the driving pin 218 projected outside from handle housing 224. A specific gear ratio has been maintained at the bevel gear 210. When knob 204 is rotated, torque is transferred to the bending section 112 for desired articulation through differential gear mechanism 406 housed in the tube assembly 102b.

Referring to FIG. 3, other embodiment for handle assembly is illustrated. The handle assembly 102a comprises a motor encoder/stepper motor for articulation. The handle assembly 102a comprises a casing 302 having a knob 304 adapted to control the articulation of a bending section 112 at desired angle with the help of a motor mechanism 306. The knob 304 at the casing 302 is connected with a rotary encoder 308 in handle housing 322. When knob 304 is rotated from its position between its specified angle range, feedback of this movement is given to motor encoder/stepper motor 310. According to the feedback, the motor encoder/stepper motor 310 rotates either in clockwise or anti-clockwise direction. The casing 302 comprises a socket 314 at a distal end of the handle assembly 102a. The socket is cylindrical shaped. The socket 314 comprises a plurality of pogo pins 316 and a driving pin 318. Each of the pogo pin from the plurality of pogo pins 316 is a spring loaded electrical connection pin. The driving pin 318 is a projection in the shape of a tapered hexagon. The shaft 312 of the motor 310 is connected with driving pin 318 projected outside from handle housing 322. The driving pin 318 is engaged with the meter pinion 410 present in a tube assembly 102(b) by inserting it into the tapered hexagonal cavity 414. When knob 304 is rotated, torque is transferred to the bending section 112 for desired articulation through differential gear mechanism 406.

Further, the socket comprises a geometry 318 adapted to lock with the tube assembly 102b. The tube assembly 102b comprises spring based locking mechanism that helps to easily attach and detach the handle assembly 102a and the tube assembly 102b. The casing 302 further comprises a PCB with RF shielding 320.

The ureteroscope 100 has a coupling mechanism such that the handle assembly 102a and the tube assembly 102b can be mutually attached and detached from each other wherein the coupling mechanism establishes an electrical interface/connection between the internal components of the handle assembly 102a and internal components of the tube assembly 102b. The lock 424 is easy to use, and is activated by pressing the push button against the spring load, which in turn creates the suitable space for a handle male geometry in the tube assembly 102b. When the handle comes at the desired location, push button is released. The spring on the button provides sufficient force that helps to engage the geometry present on the push button with the geometry on the handle. This arrangement locks the handle assembly 102a and the tube assembly 102b. The lock ensures firm attachment and proper functioning during usage.

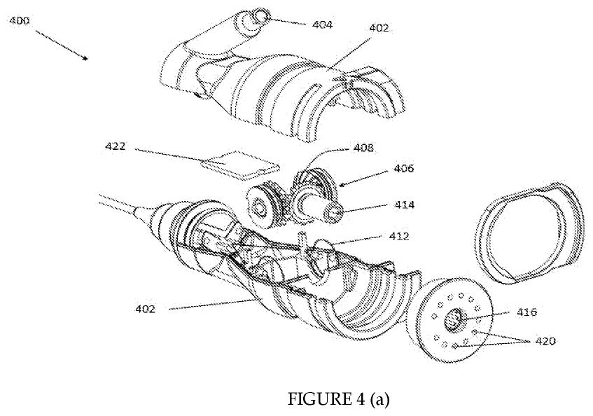

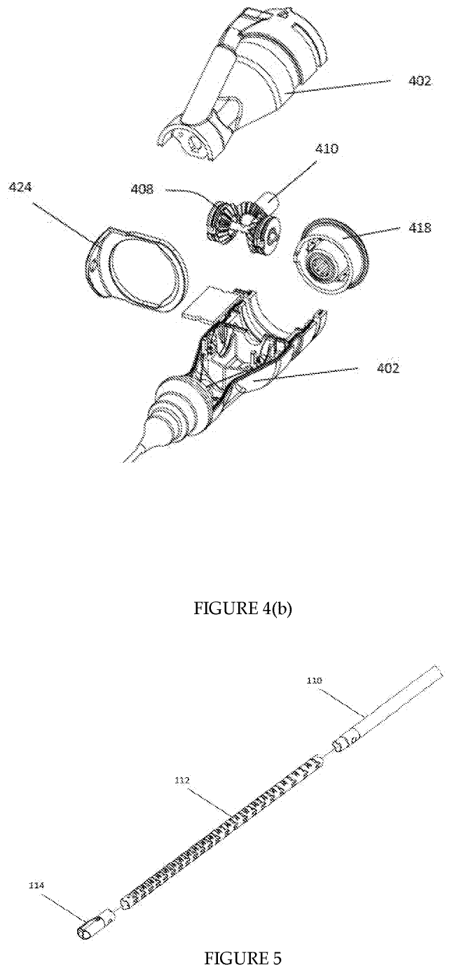

FIG. 4(a) and 4(b) illustrate the exploded view 400 of tube assembly 102b. The tube assembly 102b comprises a tube casing 402 with a working channel 404. The insertion tube casing 402 houses a differential gear mechanism 406 having a pair of meter gears 408 and meter pinion 410 that is connected with the plurality of wires 412. The plurality of wires 412 is made up of steel. One end of each of the wires 412 from the plurality of wires is welded with the bending section 112. The plurality of wires 412 passes through the insertion tube 110 and is being wound around the pair of gears 408 on the other end. One end of the meter pinion 410 comprises a tapered hexagonal cavity 414. The other end of the meter pinion 410 is engaged with pair of meter gears 408. The driving pin's hexagonal tapered projection 212 from the handle assembly 102a is positively coupled with the tapered hexagonal cavity 414 in the meter pinion 410 via a bearing 416 adapted to be at the centre of a pogo pin connector 418. The pogo pin connector 418 comprises same number of contact pads 420 as that of plurality of pogo pins 316 present on the handle assembly 102a to transfer the electrical signals. The insertion tube casing 402 also comprises a spring based lock that helps in quick engagement and disengagement of the handle assembly 102a.

The contact pads 420 are connected to a timer PCB 422 which is directly connected to the camera and the pressure sensor. The insertion tube casing 402 also comprises a spring based lock 424 that helps in quick engagement and disengagement of the ureteroscope handle assembly 102. The main function of electromechanical coupling is to transfer mechanical motion and electronic signals for image, illumination, and pressure.

The components of ureteroscope 102 may be formed of any combination of a medical based polymer, metal or other known material, as known to those of skill in the art.

Referring to FIG. 5, a disassembled view of tube assembly 500 is disclosed comprising an insertion tube 110, a bending section 112 and a distal tip 114.

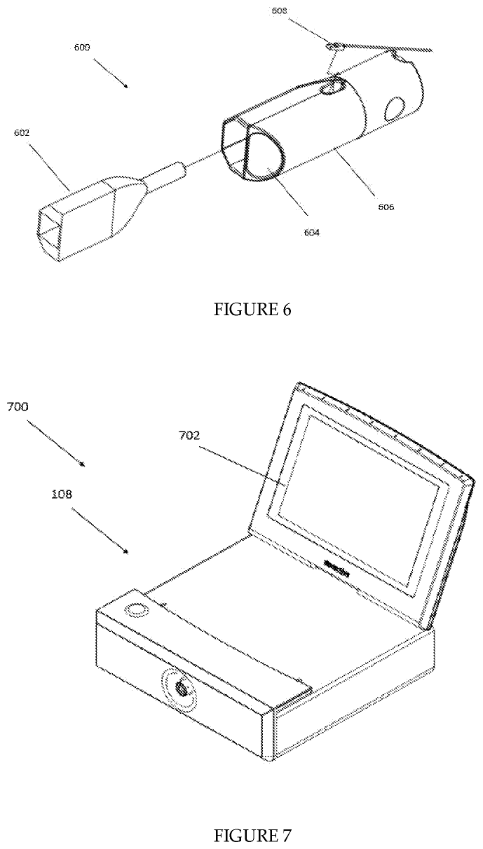

Referring to FIG. 6, an exploded view of distal tip 600 is disclosed. The distal tip 114 comprises a smallest trapezoidal shaped camera and led module arrangement 602 that helps in building a 7.5 Fr endoscope, a camera tip 604a working channel passage 606, and a pressure sensor 608 on the camera tip 604. The pressure sensor 608 and the camera 602 are connected to the Timer PCB 422 present in the tube assembly 102b by electrical wires. When the tube assembly 102b and the handle assembly 102a are attached to each other, pogo pins 318 and contact pads 420 come in contact and the image signal from the camera 602 and pressure sensor 608 are transferred.

FIG. 7 discloses the perspective view 700 of Vision Processing Unit (VPU) 108 that comprises a receptacle PCB (not shown) to receive one or more images from the camera in RGB format then performs combination of image processing steps to enhance the image quality. Dedicated image processing devices are typically used to deliver optimal image quality at high resolution with minimal lag. The VPU 108 also comprises a processor board (not shown) is employed to manage data flow and to control the algorithms and functions of the PCB. The VPU 108 further comprises a screen 702 mainly used to display the image or video captured by camera. It also has some options to control white balance, sharpness etc. It is also common to have multiple display monitors for a single system, where one or multiple display monitors are connected for viewing or control by Surgeons.

FIG. 1 to 7 are mere examples. A person skilled in the art will recognize many variations, alternatives, and modifications of the embodiment of the present disclosure.

The present invention eliminates the repeated use of the ureteroscope as the disposable tube assembly is detached which reduces the infection rate. The disposable part helps in reducing the carbon footprints as well as helps in reducing cost of surgery as the sterilization cost is reduced.

The present invention has both technical as well as economic significance with respect to the conventional ureteroscope or the like.

While a particular embodiment of the invention has been illustrated and described, modifications thereof will readily occur to those skilled in the art. It is understood that the various embodiment, details and constructions of the ureteroscope and their features described above and illustrated in the attached figures may be interchanged among the various embodiment while remaining within the scope of the invention. Additionally, it is understood that various modifications could be made to any of the partial disposable ureteroscope and/or elements described herein above while remaining within the scope of the invention.

Exemplary embodiments discussed above may provide certain advantages. Though not required to practice aspects of the disclosure, these following advantages may include:

Some embodiments of the ureteroscope provide reduction in cost and material wastage by including partially reusable and partially disposable components.

Some embodiments of ureteroscope provides a ureteroscope to solve the problem of high renal pressure in the kidney by placing sensor to control the parameters.

Although the description provides implementations of an ureteroscope, it is to be understood that the above descriptions are not necessarily limited to the specific features or methods of systems. Rather, the specific features and methods are disclosed as examples of implementations for the ureteroscope.

1. A ureteroscope (100) comprises:
a handle assembly (102a), wherein the handle assembly (102a) comprises:
a casing (302), wherein the casing (302) comprises a knob (304) and a rotary encoder (308) connected to the knob (304);
a motor (310), wherein one end of the motor (310) is connected to the rotary encoder (308) and other end of the motor (310) is connected with a driving pin (318) via a shaft (312);
a tube assembly (102b) coupled to the handle assembly (102a), wherein the tube assembly (102b) comprises:
a differential gear mechanism (406), wherein the differential gear mechanism (406) comprises a pair of meter gears (408) and a meter pinion (410), wherein the meter pinion (410) is engaged with the driving pin (318);
a plurality of wires (412), wherein a first end of each of the wires (412) is connected with the pair of meter gears (408), and wherein a second end of each of the wire is connected with a bending section (112);
wherein, during an operation, the bending section (112) is articulated based on a rotation of the knob (304).

2. The ureteroscope (100) as claimed in claim 1, wherein the handle assembly (102a) comprises a socket (314), wherein the socket (314) comprises a plurality of connection pins (316) and the driving pin (318).

3. The ureteroscope (100) as claimed in claim 2, wherein the socket (314) comprises the driving pin (318) adapted to engage with the tube assembly (102b).

4. The ureteroscope (100) as claimed in claim 1, wherein the tube assembly (102b) comprises a lock (424) and a push button configured to attach and detach the handle assembly (102a) and the tube assembly (102b), wherein during attaching the handle assembly (102a) and the tube assembly (102b), the push button is released, thereby engaging a geometry on the push button with the geometry on the handle assembly (102a).

5. The ureteroscope (100) as claimed in claim 1, wherein one end of the meter pinion (410) comprises a tapered hexagonal cavity (414), wherein the driving pin (318) is engaged with the meter pinion (410) by inserting the driving pin (318) into the tapered hexagonal cavity

6. The ureteroscope (100) as claimed in claim 1, wherein the differential gear mechanism (406) is configured to provide positional locking, thereby keeping the bending section (112) at same position when articulated.

7. The ureteroscope (100) as claimed in claim 1 comprises a Vision Processing Unit (VPU) (108) and a camera (602) coupled to the VPU (108), wherein the VPU (108) is configured to perform an image processing on one or more images received from the camera (602).

8. The ureteroscope (100) as claimed in claim 1 comprises a cable (104), wherein one end of the cable (104) is connected to the handle assembly (102a) and other end of the cable (104) is connected to the VPU (108) via a connector (106).

9. The ureteroscope (100) as claimed in claim 1, wherein the casing (302) further comprises a Printed Circuit Board (PCB) with RF shielding (320).
